(19) United States
(12) Patent Application Publication
(10) Pub. No.: US 2019/0029540 A1
(43) Pub. Date: Jan. 31, 2019

(54) MEASUREMENT DEVICE

(71) Applicant: KYOCERA CORPORATION, Kyoto (JP)

(72) Inventors: Tetsuya NEGISHI, Kawasaki-shi, Kanagawa (JP); Osamu TOCHIKUBO, Yokohama-shi, Kanagawa (JP); Hisaya HAGIWARA, Chofu-shi, Tokyo (JP)

(21) Appl. No.: 15/768,451

(22) PCT Filed: Sep. 21, 2016
(86) PCT No.: PCT/JP2016/004304

§ 371 Date: Sep. 19, 2018

(30) Foreign Application Priority Data
Oct. 14, 2015 (JP) 2015-203094

(51) Int. Cl. A61B 5/022; A61B 5/145; A61B 5/026; A61B 5/00

(52) U.S. Cl. CPC A61B 5/02241; A61B 5/14546; G01N 33/49; A61B 5/6815; A61B 5/0261

(57) ABSTRACT

A measurement apparatus includes an insertion portion insertable in an external ear canal, a pressing portion that presses a concha when the insertion portion is inserted in the external ear canal, a contact portion that comes into contact with a tragus to clamp the tragus when the concha is pressed by the pressing portion, a sensor that is mounted on the contact portion and acquires biological measurement output at the tragus when the contact portion is in contact with the tragus, and a controller that measures biological information on the basis of the biological measurement output acquired by the sensor.

### CROSS-REFERENCE TO RELATED APPLICATION

The present application claims priority to and the benefit of Japanese Patent Application No. 2015-203094 filed Oct. 14, 2015, the entire contents of which are incorporated herein by reference.

### TECHNICAL FIELD

The present disclosure relates to a measurement apparatus.

### BACKGROUND

A measurement apparatus that acquires biological measurement output using the tragus of a subject (user) as a measured part and measures biological information, such as blood pressure, on the basis of the biological measurement output is known. For example, patent literature (PTL) 1 and 2 disclose a blood pressure measurement apparatus that acquires biological measurement output from the tragus and measures the subject's blood pressure on the basis of the biological measurement output. As a method for calculating blood pressure on the basis of biological measurement output, PTL 3, for example, discloses a method for calculating blood pressure using Poiseuille's equation.

### CITATION LIST

### Patent Literature

PTL 1: JP2008-114037A

PTL 2: JP2006-288644A

PTL 3: JP2004-154231A

### SUMMARY

A measurement apparatus according to the present disclosure includes an insertion portion, a pressing portion, a contact portion, a sensor, and a controller. The insertion portion is insertable in an external ear canal. The pressing portion presses a concha when the insertion portion is inserted in the external ear canal. The contact portion comes into contact with a tragus to clamp the tragus when the concha is pressed by the pressing portion. The sensor is mounted on the contact portion and acquires biological measurement output at the tragus when the contact portion is in contact with the tragus. The controller measures biological information on the basis of the biological measurement output acquired by the sensor.

### BRIEF DESCRIPTION OF THE DRAWINGS

In the accompanying drawings:

FIG. 1 is an external perspective view, from one direction, of a measurement apparatus according an embodiment of the present disclosure;

FIG. 2 is an external perspective view, from another direction, of the measurement apparatus in FIG. 1;

FIG. 3 illustrates the holding state of a measurement mechanism in the left ear when the measurement apparatus in FIG. 1 is worn;

FIG. 4 illustrates the holding state in FIG. 3 viewed from the top of the head;

FIG. 5 is a functional block diagram illustrating the schematic configuration of the measurement apparatus in FIG. 1;

FIG. 6 is a flowchart illustrating an example of a correction coefficient calculation process in a controller;

FIG. 7 schematically illustrates the pulsatile blood flow wave height within a waveform indicating blood flow;

FIG. 8 is a flowchart illustrating an example of a blood pressure calculation process in the controller;

FIG. 9 is a schematic view of a modification, illustrating a cover mounted on the measurement apparatus in FIG. 1;

FIGS. 10A and 10B are schematic views of the cover in FIG. 9;

FIG. 11 is a schematic view of a modification to the measurement apparatus in FIG. 1;

FIG. 12 is a schematic perspective view of a modification to the measurement apparatus in FIG. 1; and

FIGS. 13A and 13B are schematic views of a modification to the measurement apparatus in FIG. 1.

### DETAILED DESCRIPTION

When measuring biological information with a measurement apparatus, more accurate measurement data can be acquired if the positional relationship between the sensor for acquiring biological measurement output and the measured part does not change during acquisition of the biological measurement output. To reduce the likelihood of a change in the positional relationship between the sensor and the measured part, PTL 1 discloses adjusting the pressure of the cuff that clamps the tragus, and PTL 2 discloses using a screw member or the like to adjust the position of the cuff that clamps the tragus. The present disclosure can improve the usefulness of a measurement apparatus.

An embodiment of the present disclosure is described below in detail with reference to the drawings.

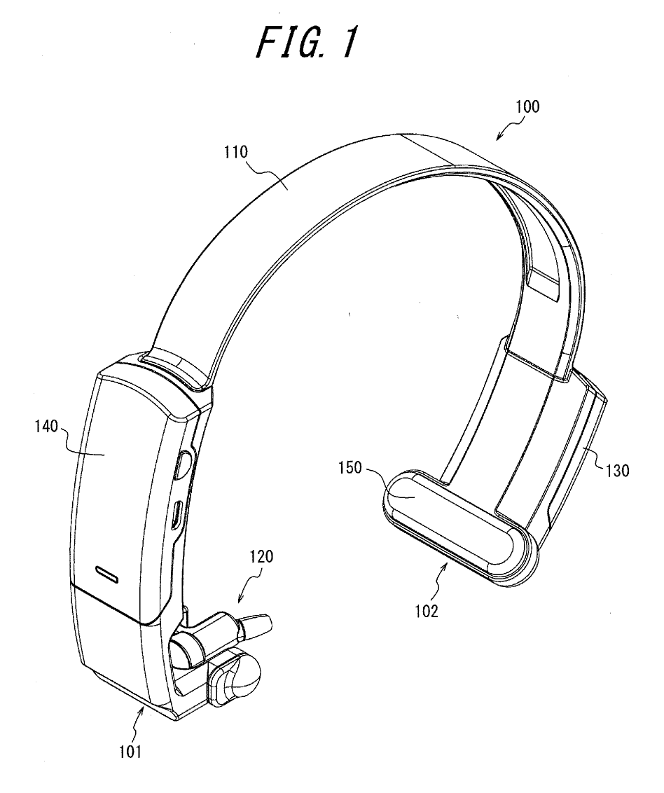

FIG. 1 is an external perspective view, from one direction, of a measurement apparatus according an embodiment of the present disclosure.

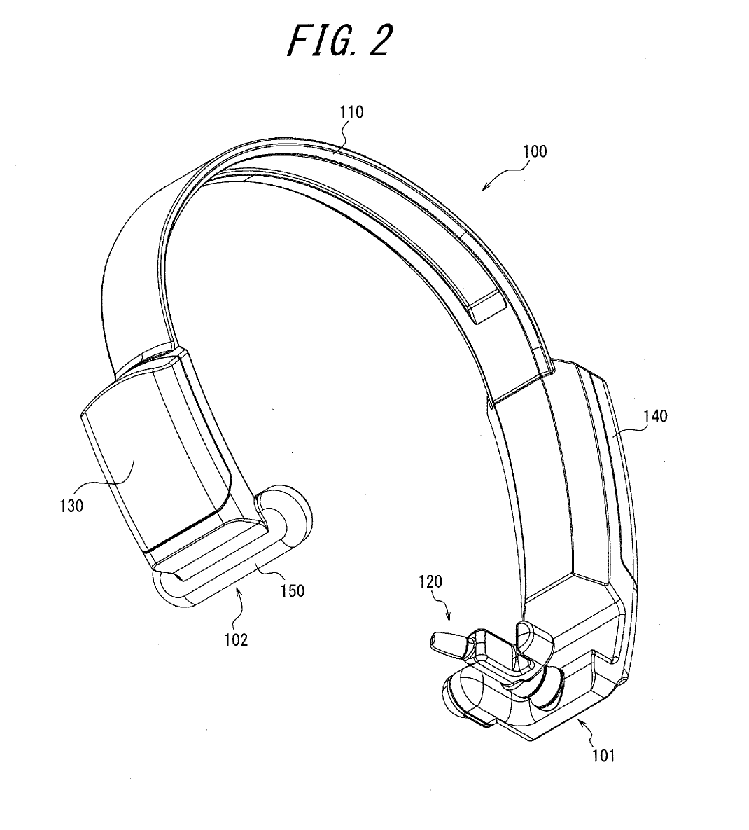

FIG. 2 is an external perspective view, from another direction, of the measurement apparatus in FIG. 1. Specifically, FIG. 2 is an external perspective view from a viewpoint in the opposite direction from the viewpoint of the external perspective view in FIG. 1.

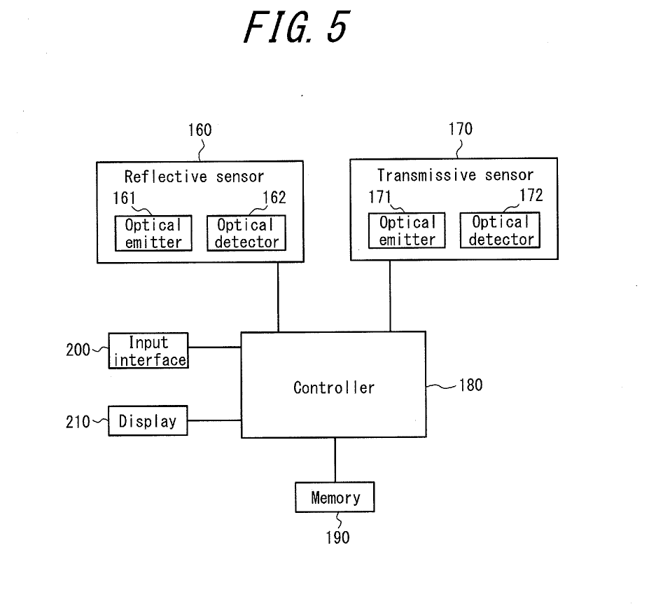

A measurement apparatus 100 includes a holder 110, a measurement mechanism 120, and a power supply holder 130. The holder 110 is an arched member capable of clamping a subject's head from the left and right. The measurement mechanism 120 is located on a first end 101 side of the holder 110. The power supply holder 130 is located on a second end 102 side, which is opposite from the first end 101 side where the measurement mechanism 120 is located. The measurement apparatus 100 includes a control mechanism holder 140 on the first end 101 side. The control mechanism holder 140 holds a control mechanism for controlling the functional blocks included in the measurement apparatus 100. Details of the functional blocks included in the measurement apparatus 100 are provided in the description of FIG. 5.

The subject wears the measurement apparatus 100 by holding the measurement mechanism 120 in the left ear, placing an abutment 150 provided on the second end 102 side in contact with the upper portion of the right ear, and placing the holder 110 over the top of the head. The abutment 150 may be attached to the holder 110 by a mechanism capable of displacement (expansion and contraction) by sliding along the holder 110. This structure allows the length from the first end 101 to the second end 102 to be changed in accordance with the size of the subject's head.

The subject measures biological information while wearing the measurement apparatus 100. For example, the measurement apparatus 100 may acquire biological measurement output with the measurement mechanism 120 in contact with the left ear and measure (calculate) biological information on the basis of the biological measurement output. The subject may continually wear the measurement apparatus 100 to continually measure biological information. In an embodiment, the measurement apparatus 100 may calculate the amount of blood flow and amount of arterial hemoglobin on the basis of the acquired biological measurement output and measure the blood pressure as biological information on the basis of the calculated amount of blood flow and amount of arterial hemoglobin. The amount of arterial hemoglobin refers to the amount of hemoglobin flowing in the arteries.

The power supply holder 130 holds a power supply for supplying electric power to the functional blocks of the measurement apparatus 100. Provision of the power supply holder 130 on the second end 102 opposite the measurement mechanism 120 facilitates an equal left-right weight balance when the subject wears the measurement apparatus 100. This facilitates stable maintenance of the wearing state.

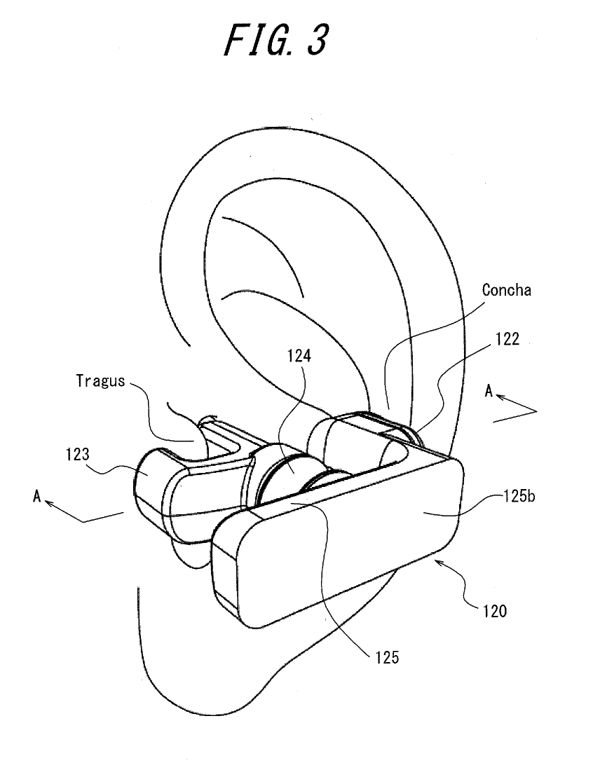

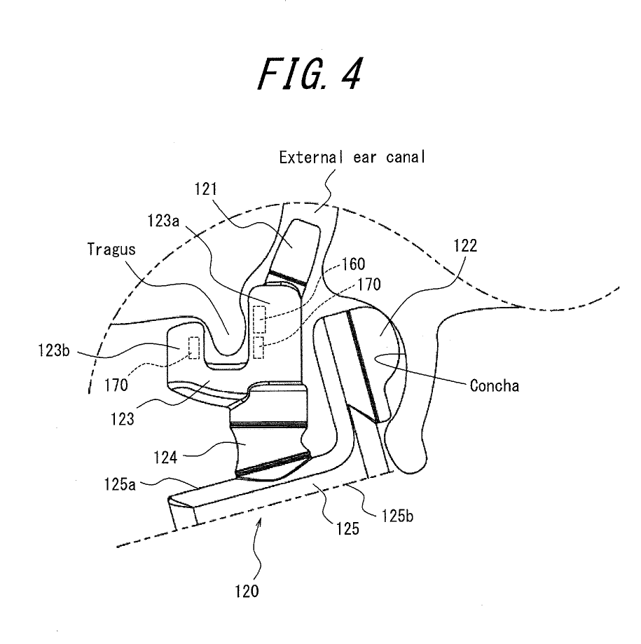

The measurement mechanism 120 acquires biological measurement output from the measured part while abutted against the measured part. Details of the measurement mechanism 120 are provided with reference to FIG. 3 and FIG. 4. FIG. 3 illustrates the holding state of the measurement mechanism 120 in the left ear when the subject wears the measurement apparatus 100 of FIG. 1. FIG. 4 illustrates the holding state in FIG. 3 viewed from the top of the head. FIG. 4 includes a cross-sectional view along the A-A line of the left ear in FIG. 3. To facilitate understanding of the measurement mechanism 120, constituent elements of the measurement apparatus 100 other than the measurement mechanism 120 are omitted from FIG. 3 and FIG. 4. For example, as illustrated in FIG. 1 and FIG. 2, the control mechanism holder 140 and holder 110 are formed on the upper head side of a frame 125 illustrated in FIG. 3 but are omitted from FIG. 3. In the present disclosure, a view from the top of the head is also referred to as a top view.

The measurement mechanism 120 includes an insertion portion 121, a pressing portion 122, a contact portion 123, and a connector 124.

The insertion portion 121 is inserted inside the left external ear canal when the subject wears the measurement apparatus 100. In other words, the subject wears the measurement apparatus 100 by holding the measurement mechanism 120 to the head so that the insertion portion 121 is inserted inside the left external ear canal.

The pressing portion 122 abuts against the concha and presses the concha towards the back of the head when the subject is wearing the measurement apparatus 100, i.e. with the insertion portion 121 inserted in the external ear canal. By the concha being pressed towards the back of the head, the distal end of the tragus stands in a direction along the external ear canal in the opposite direction from the external ear canal, i.e. in a direction towards the face. The tragus is thus more easily clamped by the contact portion 123.

The contact portion 123 is a concave member. The contact portion 123 includes two projections 123a and 123b. The projection 123a is positioned towards the back of the head when the subject wears the measurement apparatus 100. The projection 123b is positioned towards the front of the head when the subject wears the measurement apparatus 100. When the subject wears the measurement apparatus 100, the contact portion 123 comes into contact with the tragus so as to clamp the tragus with a concave recess formed between the two projections 123a and 123b. The insertion portion 121 is fixed to the distal end of the projection 123a, i.e. to the side positioned towards the head when the subject wears the measurement apparatus 100. The proximal end opposite the distal end is connected to the connector 124. In other words, the pressing portion 122 and the contact portion 123 are connected via the connector 124.

The contact portion 123 includes a sensor for optically acquiring biological measurement output. In an embodiment, the contact portion 123 includes a reflective sensor 160 and a transmissive sensor 170. Both an optical emitter and an optical detector of the reflective sensor 160 are located in the projection 123a. An optical emitter and an optical detector of the transmissive sensor 170 are located in the projections 123a and 123b respectively. The positions of the reflective sensor 160 and the transmissive sensor 170 in the contact portion 123 are indicated virtually by dotted lines in FIG. 4. The reflective sensor 160 and the transmissive sensor 170 are actually mounted inside the contact portion 123.

The reflective sensor 160 and the transmissive sensor 170 acquire biological measurement output at the tragus (measured part) of the subject. Details of the method for acquiring biological measurement output with the reflective sensor 160 and the transmissive sensor 170 are provided below.

The connector 124 connects the pressing portion 122 and the contact portion 123. In an embodiment, the contact portion 123 is connected directly to the connector 124 at the proximal end, as illustrated in FIG. 3 and FIG. 4. In an embodiment, the pressing portion 122 is connected to the connector 124 via the frame 125 on the first end 101 side of the measurement apparatus 100, as illustrated in FIG. 3 and FIG. 4. The connector 124 is formed by a movable member capable of changing the relative positional relationship between the pressing portion 122 and the contact portion 123. In an embodiment, the connector 124 is formed by an elastic member, such as rubber. The connector 124 may be formed by a material capable of changing the relative positional relationship between the pressing portion 122 and the contact portion 123. The material of the connector 124 may, for example, be a spring, resin, plastic, cloth, fiber, or the like. The connector 124 may be configured to be capable of changing the relative positional relationship between the pressing portion 122 and the contact portion 123 by a mechanical structure. Examples of a mechanical structure include a mechanism allowing the connector 124 to move using gears or the like.

The contact portion 123 is displaceable relative to the frame 125 via the connector 124. The relative positional relationship between the pressing portion 122 and the contact portion 123 changes by the contact portion 123 being displaced relative to the frame 125. The contact portion 123 is displaced relative to the frame 125 by this structure of the connector 124. Therefore, the contact portion 123 clamps the tragus to facilitate contact with the tragus regardless of the shape of the ear, in particular the positional relationship between the concha and the tragus. In the example illustrated in FIG. 4, the contact portion 123 is inclined approximately 30° towards the back of the head relative to a perpendicular to a flat portion 125a included in the frame 125 where the connector 124 is formed.

As illustrated in FIG. 3, the frame 125 includes the flat portion 125a that faces the outside of the external ear canal when the measurement apparatus 100 is worn on the ear. The connector 124 is formed on the flat portion 125a of the frame 125 at a position corresponding substantially to the center of the surface of the opposite side 125b. Since the connector 124 does not deform while the measurement apparatus 100 is not being worn on the ear, the connector 124 is formed in a substantially perpendicular direction to the opposite side 125b opposite the flat portion 125a of the frame 125. The frame 125 allows the user to easily recognize the position of the connector 124 when wearing the measurement apparatus 100 on the ear. Hence, the user can easily insert the insertion portion 121 formed at the tip of the connector 124 into the external ear canal and mount the contact portion 123 on the tragus.

FIG. 5 is a functional block diagram schematically illustrating the structure of the measurement apparatus 100. The measurement apparatus 100 includes the reflective sensor 160, the transmissive sensor 170, a controller 180, a memory 190, an input interface 200, and a display 210. The reflective sensor 160 and the transmissive sensor 170 are mounted inside the contact portion 123, as described above. The controller 180 and the memory 190 are mounted in the control mechanism holder 140. The input interface 200 and the display 210 are, for example, mounted in the power supply holder 130 or the control mechanism holder 140.

The controller 180 is a processor for controlling and managing the measurement apparatus 100 overall, starting with the functional blocks of the measurement apparatus 100. The controller 180 is a processor such as a central processing unit (CPU) that executes a program with prescribed control procedures. Such a program may, for example, be stored in the memory 190 or on an external storage medium or the like connected to the measurement apparatus 100.

The controller 180 measures the blood pressure, which is biological information, on the basis of the biological measurement output acquired by the reflective sensor 160 and the transmissive sensor 170. Details of the processing executed by the controller 180 to calculate the blood pressure are provided below.

The reflective sensor 160 irradiates the tragus with a measuring beam, acquires reflected light (scattered light) from tissue inside the tragus, and transmits a photoelectric conversion signal of the acquired scattered light to the controller 180 as biological measurement output. The reflective sensor 160 is provided with an optical emitter 161 and an optical detector 162.

The optical emitter 161 emits laser light in response to control by the controller 180. The optical emitter 161 is, for example, configured to irradiate the measured part with laser light, as a measuring beam, that has a wavelength capable of detecting a predetermined component included in blood. The optical emitter 161 is, for example, a single laser diode (LD).

The optical detector 162 detects scattered light of the measurement beam from the measured part as biological information. The optical detector 162 may, for example, be a photodiode (PD). The reflective sensor 160 transmits a photoelectric conversion signal of the scattered light detected by the optical detector 162 to the controller 180 as biological measurement output.

The controller 180 calculates the amount of blood flow at the measured part on the basis of the biological measurement output received from the reflective sensor 160. A technique for the controller 180 to measure the amount of blood flow using the Doppler shift is now described.

In the body tissue, scattered light that is scattered from moving blood cells undergoes a frequency shift (Doppler shift), due to the Doppler effect, proportional to the speed of travel of the blood cells within the blood. The controller 180 detects the beat signal produced by interference between scattered light from still tissue and the scattered light from moving blood cells. The beat signal represents strength as a function of time. The controller 180 converts the beat signal into a power spectrum that represents power as a function of frequency. In the power spectrum of the beat signal, the Doppler shift frequency is proportional to the speed of blood cells. Also, the power corresponds to the amount of blood cells in the power spectrum of the beat signal. The controller 180 calculates the amount of blood flow by multiplying the power spectrum of the beat signal by the frequency and integrating.

The transmissive sensor 170 irradiates the tragus with a measuring beam, acquires transmitted light transmitted by tissue inside the tragus, and transmits a photoelectric conversion signal of the acquired transmitted light to the controller 180 as biological measurement output. The transmissive sensor 170 is provided with an optical emitter 171 and an optical detector 172.

The optical emitter 171 emits laser light in response to control by the controller 180. For example, the optical emitter 171 irradiates the measured part with laser light, as a measuring beam, that has a wavelength capable of detecting a predetermined component included in blood. The optical emitter 171 is, for example, a laser diode (LD).

As biological information, the optical detector 172 detects transmitted light from the measurement beam transmitted through the measured part. The optical detector 172 may, for example, be a photodiode (PD). The transmissive sensor 170 transmits a photoelectric conversion signal of the transmitted light detected by the optical detector 172 to the controller 180 as biological measurement output.

In an embodiment, the transmissive sensor 170 includes two LDs for irradiating the measured part with laser light of two different wavelengths. For example, the optical emitter 171 includes an LD that irradiates laser light with a wavelength of approximately 660 nm and an LD that irradiates laser light with a wavelength of approximately 940 nm.

The absorbance with respect to light in a wavelength range of approximately 940 nm is nearly equal for venous hemoglobin present in tissue and veins and arterial hemoglobin. On the other hand, the absorbance with respect to light in a wavelength range of approximately 660 nm is higher for venous hemoglobin than for arterial hemoglobin. The detected light intensity is acquired for transmitted light that passes through an organism without being absorbed by hemoglobin and is detected by the optical detector 172 when irradiating the measured part with laser light of approximately 940 nm. The detected light intensity is also acquired for transmitted light that passes through an organism without being absorbed by hemoglobin and is detected by the optical detector 172 when irradiating the measured part with laser light of approximately 660 nm. By comparing these two detected light intensities, the amount of arterial hemoglobin can be estimated from the difference in detected light intensity (or the difference in absorbance). The controller 180 calculates the amount of arterial hemoglobin in this way. In other words, the controller 180 assumes that the absorbance is proportional to the amount of arterial hemoglobin. The absorbance does not represent the amount of arterial hemoglobin as an absolute value but rather is used only as a relative indicator.

By including LDs that irradiate laser light of two different wavelengths, the measurement apparatus 100 can calculate the amount of arterial hemoglobin to a high degree of accuracy without using the substantially difficult method of measuring the amount of arterial hemoglobin by irradiating laser light only on an artery.

The memory 190 may be configured by a semiconductor memory, a magnetic memory, or the like. The memory 190 stores a variety of information, programs for causing the measurement apparatus 100 to operate, and the like. The memory 190 may also function as a working memory. The memory 190 stores, for example, the amount of blood flow and amount of arterial hemoglobin that the controller 180 has calculated on the basis of the biological measurement outputs acquired from the reflective sensor 160 and the transmissive sensor 170. The memory 190 also stores the blood pressure that the controller 180 has measured on the basis of the amount of blood flow and the amount of arterial hemoglobin. Furthermore, the memory 190 stores a reference blood pressure value input by the subject through the input interface 200. The reference blood pressure value is the diastolic blood pressure and the systolic blood pressure that serve as a reference for the controller 180 to calculate the blood pressure. For example, before measuring blood pressure using the measurement apparatus 100, the user measures the reference blood pressure value using an upper-arm blood-pressure gauge that measures the blood pressure at the upper arm using a known cuff.

The input interface 200 receives operation input from the subject. The input interface 200 is configured using operation buttons (operation keys), for example. The input interface 200 may be configured as a touch panel, a portion of the display 210 may display operation keys that accept operation input from the subject, and this portion may accept touch operation input by the subject.

The display 210 is a display device such as a liquid crystal display, an organic EL display, an inorganic EL display, or the like. For example, the display 210 displays the result of the measurement apparatus 100 measuring biological information. The display 210 can, for example, display the measurement results with a seven-segment display.

Next, details of the process for the controller 180 to calculate the blood pressure on the basis of the calculated amount of blood flow and amount of arterial hemoglobin is described.

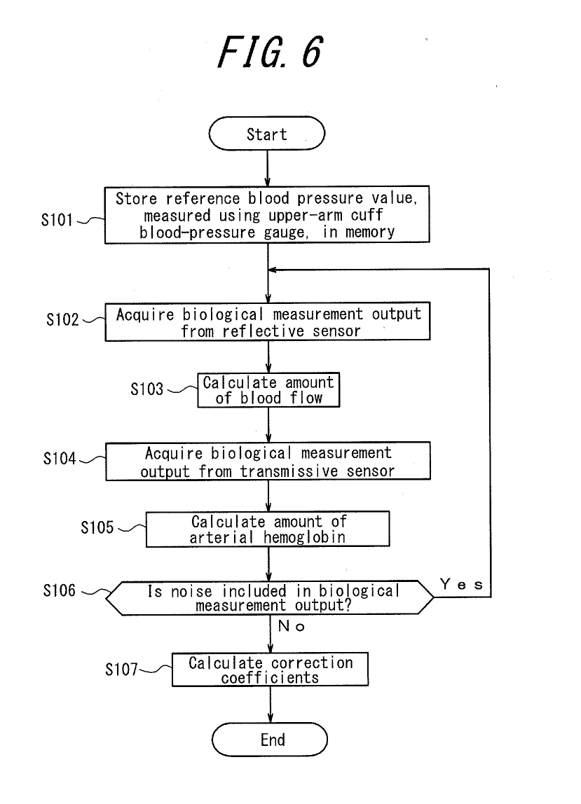

First, the controller 180 calculates a correction coefficient to use during blood pressure calculation. With reference to the flowchart in FIG. 6, an example of the correction coefficient calculation process performed by the controller 180 is described. At the start of this process, the subject inputs the reference blood pressure value using the input interface 200 and then places the measurement apparatus 100 on the head. The subject may input the reference blood pressure value after placing the measurement apparatus 100 on the head. The process in FIG. 6 is a calibration process, and the flowchart is for calculating a correction coefficient m′ and a correction coefficient θ′. The correction coefficient m′ and correction coefficient θ′ are described below.

The controller 180 stores the reference blood pressure value, which the subject measured using the upper-arm cuff blood-pressure gauge and input through the input interface 200, in the memory 190 (step S101).

Next, the controller 180 acquires the biological measurement output measured by the reflective sensor 160 from the optical detector 162 of the reflective sensor 160 (step S102).

The controller 180 calculates the amount of blood flow on the basis of the biological measurement output acquired in step S102 (step S103).

The controller 180 acquires the biological measurement output measured by the transmissive sensor 170 from the optical detector 172 of the transmissive sensor 170 (step S104).

The controller 180 calculates the amount of arterial hemoglobin on the basis of the biological measurement output acquired in step S104 (step S105).

The controller 180 need not execute step S102 through step S105 in the order indicated in FIG. 6. The controller 180 may, for example, execute step S102 and step S103 simultaneously in parallel with step S104 and step S105.

Next, the controller 180 judges whether noise is included in the biological measurement outputs acquired from the reflective sensor 160 and the transmissive sensor 170 (step S106). For example, the controller 180 judges whether noise is included in the biological measurement output on the basis of whether the period of the time change in the amount of blood flow calculated in step S103 and the period of the time change in the amount of arterial hemoglobin calculated in step S105 match. When the amount of blood flow and the amount of arterial hemoglobin exhibit a similar period of time change, the controller 180 judges that noise is not included in the biological measurement output. On the other hand, when the amount of blood flow and the amount of arterial hemoglobin exhibit different periods of time change, the controller 180 judges that noise is included in the biological measurement output.

When it is judged that noise is included in the biological measurement output (step S106: Yes), the controller 180 executes step S102 through step S105 again.

When it is judged that noise is not included in the biological measurement output (step S106: No), the controller 180 calculates the correction coefficients (step S107). The correction coefficient calculation process is thus terminated.

Here, the method executed by the controller 180 in step S107 to calculate the correction coefficients is described in detail. The controller 180 calculates the correction coefficients on the basis of the reference blood pressure value stored in the memory 190 in step S101, the calculated amount of blood flow, and the calculated amount of arterial hemoglobin.

[Measurement of Diastolic Blood Pressure DBP]

First, the method for calculating the correction coefficient (constant) m′ used in calculating the diastolic blood pressure DBPi at any time i is described. Here, the time i for calculating the correction coefficient is set to i=0. The correction coefficient m′ is the proportionality coefficient (constant) of the product of the mean amount of blood flow Q and the ratio (S0/Si) of the amounts of arterial hemoglobin when expressing the diastolic blood pressure DBPi at time i, as indicated in Expression (14) below. Here, Si is the amount of arterial hemoglobin at time i. S0 is the amount of arterial hemoglobin when the correction coefficient was calculated. In other words, for various reasons such as individual differences or individual circumstances of the sensors, the uncorrected product of the mean amount of blood flow Q and the ratio of the amounts of arterial hemoglobin (S0/Si) might not accurately indicate the diastolic blood pressure DBPi. Therefore, the diastolic blood pressure DBP is first measured at time i=0 using, for example, the auscultatory method (Korotkoff method) or an upper-arm cuff that uses the oscillometric method. In this way, the correction coefficient m′ is determined by using the measurement apparatus 100 of the present embodiment to measure the value indicating the product of the mean amount of blood flow Q measured at i=0 and the ratio (S0/Si) of amounts of arterial hemoglobin. This correction coefficient m′ might differ with various conditions, such as for each individual or each measurement apparatus 100. Hence, a process for determining m′ at the initial measurement time is necessary. The method for measuring the diastolic blood pressure DBPi at time i=0 may be any other appropriate method other than the aforementioned method using an upper-arm cuff.

The mathematical expressions that the controller 180 uses to calculate the constant m′ are Expression (1) to Expression (3) below.

$$\begin{matrix}P = Q \times R & ( 1 ) \\ P \approx DBP + \frac{SBP - DBP}{3} & ( 2 ) \\ SBP = a \times DBP + b & ( 3 )\end{matrix}$$

In Expression (1) to Expression (3), P, Q, R, DBP, and SBP respectively represent the mean blood pressure, mean amount of blood flow, vascular resistance, diastolic blood pressure, and systolic blood pressure, and a and b are constants. Expression (4) follows from Expression (1) to Expression (3).

$$P = DBP ( \frac{2}{3} + \frac{a}{3} ) + \frac{b}{3} = Q \times R \qquad ( 4 )$$

Here, m (constant) is substituted for 2/3+a/3 in Expression (4). Normally, the value of b is approximately 5 mmHg to 15 mmHg in Expression (4). Therefore, b/3 is approximately 2 mmHg to 5 mmHg. The constant b is unique for each individual. If b/3 is approximately 2 mmHg to 5 mmHg, b/3 can be approximated as follows, since b/3 can be considered to be included in m′ sought in Expression (14).

b/3≈0

Therefore, Expression (4) can be transformed into Expression (5).

Q×R=mDBP (5)

Transforming Expression (5) leads to Expression (6).

$$DBP = \frac{QR}{m} \qquad ( 6 )$$

Here, the mean amount of blood flow Q can also be expressed as in Expression (7) using the mean blood flow speed V and the arterial radius r.

Q∝V×πr² (7)

The vascular resistance R can be expressed by Poiseuille's law as in Expression (8) using the blood viscosity μ, the arterial radius r, and the blood vessel length L.

$$R = \frac{8 μL}{π {r}^{4}} \qquad ( 8 )$$

Transforming Expression (1), Expression (7), and Expression (8) yields Expression (9).

$$\begin{matrix}P = Q \times R ∝ V \times π {r}^{2} \times \frac{8 μL}{π {r}^{4}} \\ = V \times \frac{8 μL π}{π {r}^{2}} \\ = C \times \frac{V}{S}\end{matrix} \qquad ( 9 )$$

In Expression (9), S is the arterial cross-sectional area πr² and is proportional to the amount of arterial hemoglobin. C is a constant representing 8 μLπ. Expression (10) follows from Expression (9).

$$Q \times R ∝ \frac{V}{S} \qquad ( 10 )$$

Expression (11) follows from Expression (6) and Expression (10).

$$DBP ∝ \frac{1}{m} \times \frac{V}{S} \qquad ( 11 )$$

As described above, S is the arterial cross-sectional area and is proportional to the amount of arterial hemoglobin. In an embodiment, the measurement apparatus 100 uses the absorbance, acquired as biological measurement output by the transmissive sensor 170, as an indicator of the amount of arterial hemoglobin. To simplify the explanation below, S indicating the arterial cross-sectional area is also referred to as the amount of arterial hemoglobin. Using the initial amount of arterial hemoglobin S0 and the amount of arterial hemoglobin Si at any time i, S in Expression (11) can thus be replaced with the rate of change from S0 to Si to yield Expression (12) below for the diastolic blood pressure DBPi at any time i. The initial amount of arterial hemoglobin S0 is the amount of arterial hemoglobin calculated by the controller 180 on the basis of the biological measurement output (absorbance) acquired by the transmissive sensor 170 when the controller 180 calculated the correction coefficient. The amount of arterial hemoglobin Si at any time i is the amount of arterial hemoglobin calculated by the controller 180 on the basis of the biological measurement output (absorbance) acquired by the transmissive sensor 170 at time i.

$$DBPi ∝ \frac{V}{m} \times \frac{SO}{Si} \qquad ( 12 )$$

Here, the mean blood flow speed V is proportional to the mean amount of blood flow Q by the definition of the blood flow speed V, i.e. V=Q/S. Hence, Expression (12) can be transformed into Expression (13).

$$DBPi ∝ \frac{Q}{m} \times \frac{SO}{Si} \qquad ( 13 )$$

The constant m′ is the constant corresponding to the constant m in Expression (13) when the correction coefficient is calculated, i.e. when Si=S0. The constant m′ is determined by Expression (13) on the basis of the diastolic blood pressure DBP of the reference blood pressure value stored in the memory 190 and the mean amount of blood flow Q calculated by the controller 180. Consequently, the diastolic blood pressure DBPi at any time i is expressed by Expression (14) using the calculated constant m′.

$$DBPi = \frac{Q}{{m}^{′}} \times \frac{SO}{Si} \qquad ( 14 )$$

[Measurement of Systolic Blood Pressure SBPi]

Next, the method for calculating the correction coefficient (constant) θ′ used in calculating the systolic blood pressure SBPi at any time i is described. The correction coefficient θ′ is the proportionality coefficient (constant) with respect to the pulsatile blood flow wave height qpp when representing the systolic blood pressure SBP at time i, as indicated in Expression (24) below. In other words, for various reasons such as individual differences or individual circumstances of the sensors, the uncorrected pulsatile blood flow wave height qpp might not accurately indicate the systolic blood pressure SBPi. Therefore, the correction coefficient θ′ is determined by first measuring the systolic blood pressure SBP using a cuff, and at the time of this measurement, measuring the diastolic blood pressure DBP of the reference blood pressure value, the constant m′, and the pulsatile blood flow wave height qpp using the measurement apparatus 100 of the present embodiment. In other words, this correction coefficient θ′ might differ with various conditions, such as for each individual or each measurement apparatus 100. Hence, a process for determining θ′ at the initial measurement time is necessary.

Expression (15) to Expression (18) are mathematical expressions that the controller 180 uses to calculate the constant θ′.

$$\begin{matrix}Q = SV \times HR & ( 15 ) \\ qpp = \frac{Q}{HR} - Roff & ( 16 ) \\ PP = SBP - DBP = E ( SV - Roff ) & ( 17 ) \\ MBP = DBP + \frac{1}{3} ( SBP - DBP ) = QR & ( 18 )\end{matrix}$$

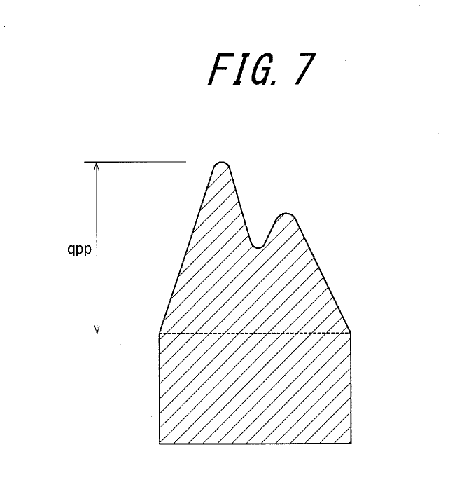

In Expression (15) to Expression (18), qpp, PP, and MBP are respectively the pulsatile blood flow wave height, pulse pressure, and mean pulse pressure. The pulse pressure is the difference between the systolic blood pressure (maximum blood pressure) and the diastolic blood pressure (minimum blood pressure). The mean blood pressure refers to the average of the blood pressure in the arteries and is calculated from the systolic blood pressure (maximum blood pressure) and the diastolic blood pressure (minimum blood pressure). The pulsatile blood flow wave height qpp is the maximum difference in the amount of blood flow in one beat, as illustrated schematically in the example in FIG. 7. The pulsatile blood flow wave height qpp is derived from the amount of blood flow calculated by the controller 180 on the basis of the biological measurement output acquired from the reflective sensor 160. SV is the stroke volume in one beat. HR is the heart rate, and Roff is the amount of blood flow flowing out from the artery during systole of the blood vessel (run off in systole). E is the elastic modulus of the pulse.

Expression (19) follows from Expression (15) and Expression (16).

qpp=SV−Roff (19)

Expression (20) follows from Expression (17) and Expression (19).

DBP=SBP−E×qpp (20)

Expression (18) can be transformed into Expression (21).

$$\frac{2}{3} DBP + \frac{1}{3} SBP = QR \qquad ( 21 )$$

Substituting Expression (20) into Expression (21) and simplifying leads to Expression (22).

$$SBP = QR + 2 E \times \frac{qpp}{3} . \qquad ( 22 )$$

Using Expression (6) and Expression (22) and setting 0=2E/3 leads to Expression (23).

SBP=m×DBP+θ×qpp (23)

The correction coefficient θ′ is the coefficient corresponding to θ calculated by substituting the diastolic blood pressure DBP and systolic blood pressure SBP of the reference blood pressure value, the above-calculated m′, and the pulsatile blood flow wave height qpp into Expression (23). The pulsatile blood flow wave height qpp is calculated from the amount of blood flow calculated by the controller 180 on the basis of the biological measurement output acquired from the reflective sensor 160. The systolic blood pressure SBPi at any time i is expressed by Expression (24), using the constant θ′ calculated in this way. In Expression (24), qppi is the pulsatile blood flow wave height at time i.

SBPi=m×DBPi+θ′×qppi (24)

The controller 180 uses the calculated correction coefficients m′ and θ′ to calculate the diastolic blood pressure DBPi and the systolic blood pressure SBPi of a subject at any time i on the basis of Expression (14) and Expression (24).

[Example of Process for Calculating Blood Pressure]

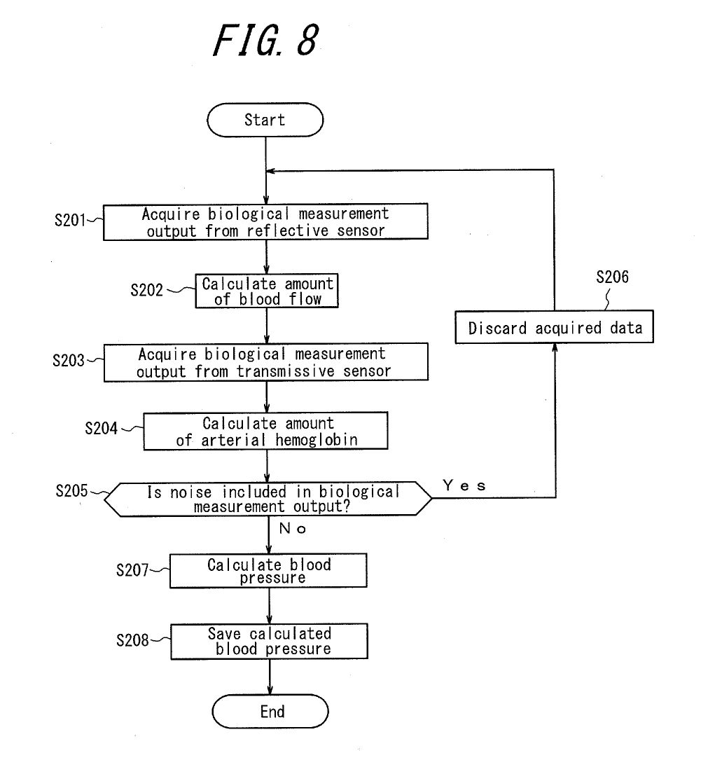

Next, with reference to the flowchart in FIG. 8, an example of the process for the controller 180 to calculate the subject's blood pressure is described. The controller 180 can perform the process to calculate blood pressure on the basis of any biological measurement output of the heart rate. For example, the controller 180 performs the process to calculate blood pressure according to the process in FIG. 8 on the basis of the biological measurement output for five beats. The controller 180 determines the five beats of the pulse on the basis of the calculated period of the amount of blood flow, for example.

First, as in steps S102 and S103 in FIG. 6, the controller 180 acquires the biological measurement output for five beats from the reflective sensor 160 (step S201) and calculates the amount of blood flow on the basis of the biological measurement output (step S202).

As in steps S104 and S105 in FIG. 6, the controller 180 also acquires the biological measurement output for five beats from the transmissive sensor 170 (step S203) and calculates the amount of arterial hemoglobin on the basis of the biological measurement output (step S204).

As in step S106 in FIG. 6, the controller 180 then judges whether noise is included in the biological measurement outputs acquired from the reflective sensor 160 and the transmissive sensor 170 (step S205).

When it is judged that noise is included in the biological measurement output (step S205: Yes), the controller 180 discards (deletes) the acquired biological measurement output data (step S206). The controller 180 then executes step S201 through step S204 again.

When it is judged that noise is not included in the biological measurement output (step S205: No), the controller 180 calculates the subject's blood pressure on the basis of Expression (14) and Expression (24) using the calculated correction coefficients m′ and θ′ (step S207).

The controller 180 saves the calculated blood pressure of the subject by storing the blood pressure in the memory 190 (step S208).

The controller 180 accumulates data related to the subject's blood pressure by repeating the process in FIG. 8. The subject, a doctor, and the like can learn of changes in the subject's blood pressure from the accumulated data.

As described above, the measurement apparatus 100 according to an embodiment is configured so that when the subject is wearing the measurement apparatus 100, the concha is pressed towards the back of the head by the pressing portion 122, so that the tragus faces away from the head. The measurement apparatus 100 can therefore clamp the tragus more easily with the contact portion 123 regardless of the shape of the subject's ear. In this way, the measurement apparatus 100 is more useful.

The contact portion 123 and pressing portion 122 of the measurement apparatus 100 are connected via the connector 124 formed by a movable member. The relative positional relationship between the pressing portion 122 and the contact portion 123 changes by the contact portion 123 being displaced relative to the frame 125 via the connector 124. Therefore, the contact portion 123 more easily comes in contact with the tragus, regardless of the shape of the subject's ear.

The measurement apparatus 100 includes the holder 110, which is an arched member capable of clamping the subject's head from the left and right. Therefore, when the subject is wearing the measurement apparatus 100, the measurement apparatus 100 holds the subject's head by applying pressure from the left and right. The contact portion 123 is thus fixed to the tragus more easily.

In the measurement apparatus 100, the power supply holder 130 is provided at the second end 102 side, on the opposite side from the measurement mechanism 120. This facilitates an equal left-right weight balance when the subject wears the measurement apparatus 100, thereby facilitating stable maintenance of the wearing state.

The measurement apparatus 100 calculates correction coefficients on the basis of known equations and the subject's reference blood pressure value input by the subject and calculates biological information on the basis of the calculated correction coefficients and biological measurement output acquired from the sensor. Therefore, the measurement apparatus 100 calculates the biological information with higher reliability and measurement accuracy than a known measurement apparatus.

The measurement apparatus 100 judges whether noise is included in the acquired biological measurement outputs on the basis of biological measurement outputs acquired by the reflective sensor 160 and the transmissive sensor 170. When it is judged that noise is included in the biological measurement outputs, the measurement apparatus 100 does not use the biological measurement outputs, but rather reacquires the biological measurement outputs. Hence, the reliability and measurement accuracy of the measured biological information improve.

The measurement apparatus 100 irradiates laser light of two different wavelengths on the measured part using the transmissive sensor 170. Therefore, the measurement apparatus 100 can compare the detected light intensities of the transmitted light detected by the optical detector 172 to estimate the amount of arterial hemoglobin from the difference therebetween. By estimating the amount of arterial hemoglobin in this way, the measurement apparatus 100 can estimate the amount of arterial hemoglobin more accurately than a known measurement apparatus.

The present disclosure is not limited to the above embodiments, and a variety of modifications and changes are possible. For example, the functions and the like included in the components, steps, and the like may be reordered in any logically consistent way. Furthermore, components, steps, and the like may be combined into one or divided.

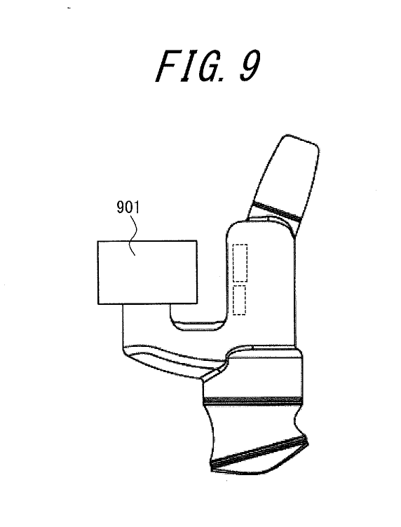

For example, as illustrated in FIG. 9, the measurement apparatus 100 may include a cover 901 over the projection 123b of the contact portion 123. FIG. 9 is a schematic view of a modification, illustrating the cover 901 mounted on the measurement apparatus 100 in FIG. 1. The cover 901 may be made of a material capable of transmitting light beams emitted from the transmissive sensor 170 so as not to block acquisition of the biological measurement output by the transmissive sensor 170. The cover 901 may be detachable from the projection 123b.

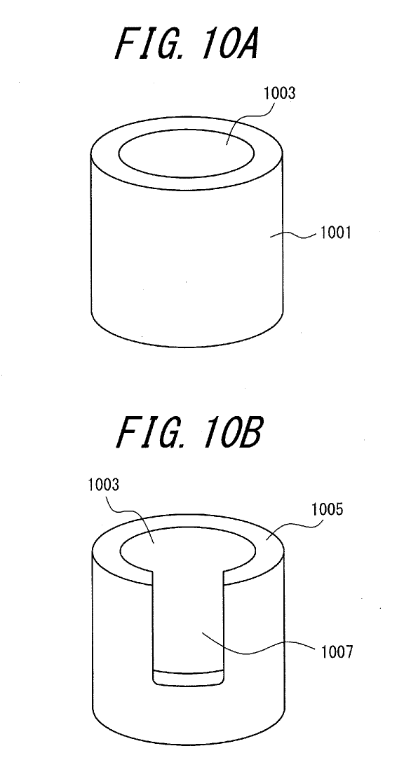

The cover 901 illustrated in FIG. 9 is further described with reference to FIGS. 10A and 10B. FIG. 10A is a schematic view of the cover 901 in FIG. 9. As illustrated in FIG. 10A, the cover 1001 includes a hole 1003 into which the projection 123b is inserted and may, for example, be formed from a material such as resin or plastic. The thickness of the projection 123b in the measurement apparatus 100 changes by the cover 1001 being attached to or removed from the projection 123b. By changing the thickness of the projection 123b in this way, the subject can adjust the contact strength of the contact portion 123 relative to the tragus in accordance with the shape and thickness of the tragus. In particular, providing a plurality of types of covers 1001 allows the subject to select the cover 1001 for the contact portion 123 to come into contact with the tragus with the most appropriate contact strength for the shape and thickness of the subject's own tragus. The appropriate contact strength refers to, for example, the contact strength at which the measurement accuracy of biological information increases, the contact strength at which the subject feels less discomfort when wearing the measurement apparatus 100, the contact strength at which the positional relationship between the tragus and the contact portion 123 tends not to change, or the like.

As illustrated in FIG. 10B, the cover 1005 may expose the transmissive sensor 170 to the outside while the cover 1005 is mounted on the contact portion 123 to avoid blocking acquisition of the biological measurement output by the transmissive sensor 170. In other words, the cover 1005 may include an opening 1007 on the tragus side for exposing the transmissive sensor 170.

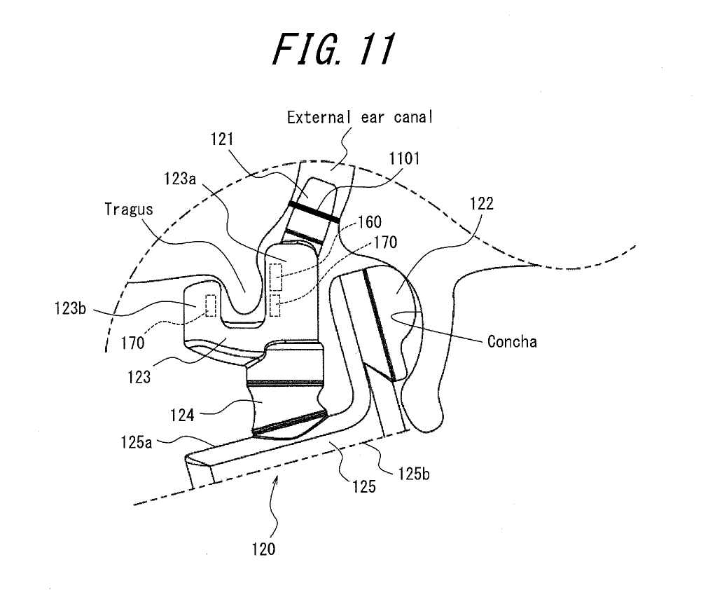

As illustrated in FIG. 11, the insertion portion 121 may include a lid 1101 covering the external ear canal when the insertion portion 121 is inserted in the external ear canal. FIG. 11 is a schematic view of a modification to the measurement apparatus 100 in FIG. 1. Inclusion of the lid 1101 allows the lid of the insertion portion 121 to come into contact with the surface of the external ear canal so that the insertion portion 121 is inserted into the external ear canal in a more stable state. The lid 1101 may be made from a material through which sound passes easily, such as sponge, rubber, cloth, plastic, or resin, so as not to block sound that the subject hears from the surroundings.

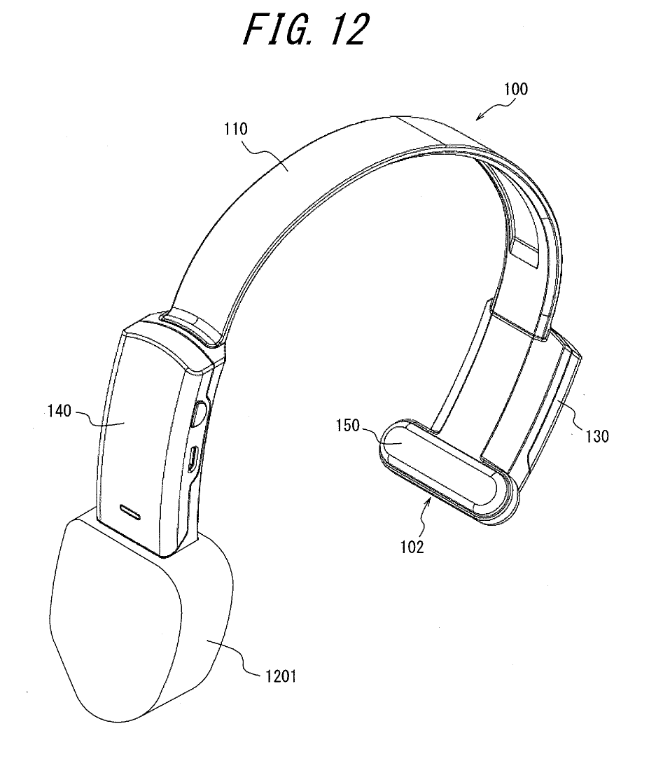

The measurement apparatus 100 may include a light-blocking portion to block external light incident on the sensor when the contact portion 123 is in contact with the tragus. FIG. 12 is a schematic perspective view of a modification to the measurement apparatus 100 in FIG. 1. For example, as illustrated in FIG. 12, a light-blocking portion 1201 may be an ear cover, formed from material such as cloth, plastic, or resin, that covers the entire auricle along with the insertion portion 121 and the reflective sensor 160 and transmissive sensor 170 of the contact portion 123.

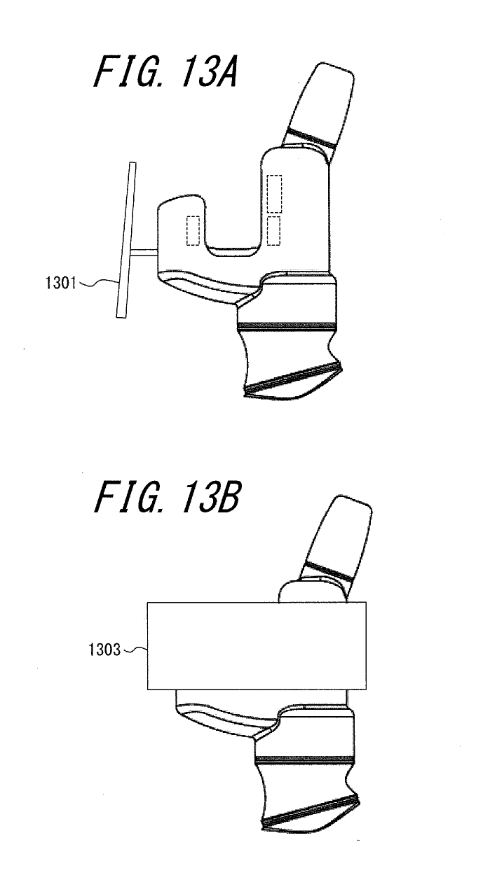

FIGS. 13A and 13B are schematic views of a modification to the measurement apparatus 100 in FIG. 1, illustrating a different type of light-blocking portion than the light-blocking portion 1201 illustrated in FIG. 12. As illustrated in FIG. 13A, a light-blocking portion 1301 may, for example, be formed on the side of the contact portion 123 facing the front of the head (towards the face) to block light entering the reflective sensor 160 and the transmissive sensor 170 from the direction of the front of the head (the face). As illustrated in FIG. 13B, a light-blocking portion 1303 may, for example, be formed on the side of the contact portion 123 facing the top of the head to block light entering the reflective sensor 160 and the transmissive sensor 170 from the direction of the top of the head above the contact portion 123. The light-blocking portions 1301 and 1303 may, for example, be screens formed from plastic, resin, or the like.

By including the light-blocking portion 1201, 1301, or 1303, the measurement apparatus 100 can block external light from entering the sensor, thereby facilitating the removal of noise that can be produced by external light when biological measurement output is acquired. The light-blocking portion may be configured as any combination of the light-blocking portions 1201, 1301, and 1303 in FIG. 12 and FIGS. 13A and 13B.

The measurement apparatus 100 may be configured so that the weight thereof is adjusted for the downward forces in the direction of gravity on the first end 101 and the second end 102 to be substantially equal when the first end 101 and the second end 102 are facing downward in the direction of gravity. This configuration makes the downward forces in the direction of gravity on the first end 101 and the second end 102 nearly equal when the measurement apparatus 100 is worn on a human head, improving the wearing performance of the measurement apparatus 100 on the head.

The measurement apparatus 100 of the above embodiment includes the arched holder 110 that clamps the subject's head from the left and right, the measurement mechanism 120 provided on the first end 101 side, and the power supply holder 130 provided on the second end 102 side opposite the first end 101 side. The present disclosure is not limited to this embodiment, however. For example, the measurement apparatus may be worn on the head by configuring the measurement mechanism 120 to include a wearing portion worn on the auricle of only one of the left and right ears. That is, a structure without a holder such as the holder 110, illustrated in FIG. 1, in the measurement apparatus 100 of the above embodiment may be adopted. By omitting a holder such as the holder 110 illustrated in FIG. 1, this structure can reduce the weight of the apparatus overall and is more convenient, since it does not disturb the user's hair.

In the measurement apparatus 100 of the above embodiment, the measurement mechanism 120, power supply holder 130, or control mechanism holder 140 may have a waterproof structure or a dustproof structure. In this case, the measurement apparatus 100 can be used even on rainy days, for example, increasing the opportunities for using the measurement apparatus 100 and increasing convenience.

The measurement apparatus 100 of the above embodiment may have a communication function that is wired, wireless, or a combination thereof. The wired communication function may be USB, LAN, or the like. The wireless communication function may be long term evolution (LTE), a wireless local area network (LAN), infrared communication, or the like. By incorporating such a communication function, the measurement apparatus 100 can, for example, be operated or controlled by an external operation terminal and can transmit various measured information to an external apparatus.

The measurement apparatus 100 of the above embodiment measures the amount of blood flow and the amount of arterial hemoglobin as biological information but may be configured to measure other types of biological information. Depending on the biological information that the measurement apparatus 100 acquires, the measurement apparatus 100 may be provided with an appropriate combination of various sensors, such as a body temperature sensor, a pulse wave sensor, a vibration sensor, a sound sensor, a humidity sensor, an altitude sensor, a direction sensor, a position sensor, or a brightness sensor.

The measurement apparatus 100 of the above embodiment incorporates the power supply holder 130. As the power source of the measurement apparatus 100, however, a power source may be separately provided in a separate housing from the measurement apparatus 100, and electric power may be supplied from the power source to the components of the measurement apparatus 100 in a wired or wireless manner:

In the above embodiment, the controller 180 provided in the measurement apparatus 100 has been described as generating the biological information on the basis of biological measurement output acquired by a sensor, but the biological information is not limited to being generated by the controller 180 provided in the measurement apparatus 100. For example, a server that is connected to the measurement apparatus 100 by a network that is wired, wireless, or a combination of both may be provided with a functional component corresponding to the controller 180, and the biological information may be generated by the server that includes this functional component. In this case, the measurement apparatus 100 transmits the biological measurement output acquired by the sensor to the server via a separately provided communication interface. The server calculates the biological information on the basis of the biological information output and stores the calculated biological information in the memory. When the server calculates and stores biological information in this way, the measurement apparatus 100 can, for example, be reduced in size as compared to when all of the functional components in FIG. 1 are implemented on one measurement apparatus 100.

### REFERENCE SIGNS LIST

100 Measurement apparatus
101 First end
102 Second end
110 Holder
120 Measurement mechanism
121 Insertion portion
122 Pressing portion
123 Contact portion
123a, 123b Projection
124 Connector
125 Frame
125a Flat portion
125b Opposite side
130 Power supply holder
140 Control mechanism holder
150 Abutment
160 Reflective sensor
161, 171 Optical emitter
162, 172 Optical detector
170 Transmissive sensor
180 Controller
190 Memory
200 Input interface
210 Display
901, 1001, 1005 Cover
1101 Lid
1201, 1301, 1303 Light-blocking portion

1. A measurement apparatus comprising:
an insertion portion insertable in an external ear canal;
a pressing portion configured to press a concha when the insertion portion is inserted in the external ear canal;
a contact portion configured to come into contact with a tragus to clamp the tragus when the concha is pressed by the pressing portion;
a sensor mounted on the contact portion and configured to acquire biological measurement output at the tragus when the contact portion is in contact with the tragus; and
a controller configured to measure biological information on the basis of the biological measurement output acquired by the sensor.

2. The measurement apparatus of claim 1, wherein the contact portion and the pressing portion are connected by a movable member capable of changing a relative positional relationship between the contact portion and the pressing portion.

3. The measurement apparatus of claim 1, wherein the contact portion comprises a detachable cover.

4. The measurement apparatus of claim 3, wherein the cover is made of a material capable of transmitting a light beam emitted from the sensor.

5. The measurement apparatus of claim 3, wherein the cover comprises an opening for exposing the sensor.

6. The measurement apparatus of claim 1, further comprising a holder configured to clamp a head from left and right.

7. The measurement apparatus of claim 6, wherein the holder comprises the contact portion on one side and a power source, configured to provide electric power to the sensor, on another side opposite the one side.

8. The measurement apparatus of claim 6, wherein the holder is configured so that downward forces in a direction of gravity on one end and another end are substantially equal when the one end and the another end are facing downward in the direction of gravity.

9. The measurement apparatus of claim 1, wherein the insertion portion comprises a lid covering the external ear canal when the insertion portion is inserted in the external ear canal.

10. The measurement apparatus of claim 1, further comprising a light-blocking portion configured to block external light incident on the sensor when the contact portion is in contact with the tragus.

11. The measurement apparatus of claim 10, wherein the light-blocking portion is an ear cover covering an entire auricle.

12. The measurement apparatus of claim 10, wherein the light-blocking portion is formed on a side of the contact portion facing a front of a head.

13. The measurement apparatus of claim 10, wherein the light-blocking portion is formed on a side of the contact portion facing a top of a head.

14. The measurement apparatus of claim 1, wherein the pressing portion presses the concha towards a back of a head.

15. The measurement apparatus of claim 2,
further comprising a frame in which the pressing portion and the movable member are formed,
wherein the frame comprises a flat portion facing an outside of the external ear canal and the movable member is formed on the flat portion substantially at a center of an opposite side of the flat portion.
